(19) United States
(12) Patent Application Publication
(10) Pub. No.: US 2012/0039972 A1
(43) Pub. Date: Feb. 16, 2012

(54) OILY COMPOSITION AND ROD-SHAPED PREPARATION

(75) Inventors: Yosuke Kobayashi, Osaka-shi (JP); Akiyori Fujiwara, Osaka-shi (JP); Takashi Wada, Tondabayashi-shi (JP); Atsushi Seto, Tondabayashi-shi (JP)

(73) Assignee: ROHTO PHARMACEUTICAL CO., LTD., Osaka-shi, Osaka (JP)

(21) Appl. No.: 13/264,134

(22) PCT Filed: Apr. 28, 2010
(86) PCT No.: PCT/JP2010/057549

§ 371 Date: Oct. 12, 2011

(30) Foreign Application Priority Data
May 8, 2009 (JP) 2009-113671
May 8, 2009 (JP) 2009-113675

(51) Int. Cl. A61K 8/02; A61P 17/00; A61K 8/88; A61Q 19/00; A61K 47/34; A61K 8/92

(52) U.S. Cl. 424/401; 514/772.3; 424/400

(57) ABSTRACT

An oily composition which gives a preparation for lip through solidification, characterized by comprising: (A) an N-acylamino acid derivative containing, for example, two N-acylglutamic acid dialkylamide; (B) a polyamide resin containing, for example, (dimer dilinoleic acid bisdioctadecylamide/ethylenediamine) copolymer; and (C) a liquid oil containing, for example, dimer acid esters; and which, upon solidification to give a preparation for lip, can exhibit sufficient transparency and further can give a satisfactory use feeling.

### TECHNICAL FIELD

The present invention relates to an oily composition which gives a rod-shaped preparation for lip through solidification, and the rod-shaped preparation.

### BACKGROUND ART

Patent document 1 has a description related to an example of a transparent lip rouge in the form of a rod-shaped preparation (see [0044], [0045]).

Patent document 1: JP-A-2002-316971

### DISCLOSURE OF THE INVENTION

### Problem to be Solved by the Invention

Patent document 1 has, however, utterly no description regarding the extent of transparency and a use feeling as a lip rouge.

Rod-shaped preparations for lip, particularly, lip rouges are required to have high transparency from the standpoint of apparent beauty and a luster feeling in use. However, the example in patent document 1 has a possibility of no sufficient transparency.

In addition, lip has a very keen feeling as compared with other skins and feels even a slight change as uncomfortable, thus leading to a special desire for a preparation having an excellent use feeling.

The present invention has an object of providing an oily composition which, upon solidification to give a rod-shaped preparation for lip, can exhibit sufficient transparency and further can give a satisfactory use feeling, and providing the rod-shaped preparation.

### Means for Solving the Problem

The present invention relates to an oily composition which gives a preparation for lip through solidification, characterized by comprising:

(A) an N-acylamino acid derivative represented by the following formula (1),

(B) a polyamide resin, and

(C) a liquid oil.

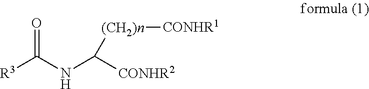

In the formula (1), R¹ and R² represent each independently a hydrocarbon group having 1 to 26 carbon atoms, R³ represents a hydrocarbon group having 7 to 18 carbon atoms, and n is 1 or 2.

The rod-shaped preparation of the present invention is characterized in that it is obtained by solidification of the oily composition of the present invention into a rod-shape.

### Effect of the Invention

The oily composition of the present invention is, upon solidification to give a rod-shaped preparation for lip, capable of exhibiting sufficient transparency. Thus, apparent beauty and a luster feeling in use can be improved, and a product's value can be enhanced.

The rod-shaped preparation of the present invention is capable of improving apparent beauty and a luster feeling in use, and enhancing a product's value.

### BRIEF DESCRIPTION OF DRAWINGS

FIG. 1 is a longitudinal sectional view of the rod-shaped article of the present invention.

### DESCRIPTION OF REFERENCE NUMERALS

100: rod-shaped article

2: rod-shaped preparation

40: tubular container

### BEST MODES FOR CARRYING OUT THE INVENTION

(Oily Composition)

The oily composition of the present invention comprises the following components (A), (B) and (C).

[Regarding Component (A)]

The component (A) is an N-acylamino acid derivative represented by the following formula (1).

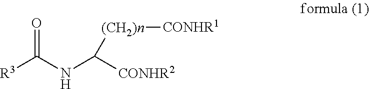

In the formula (1), R¹ and R² represent each independently a hydrocarbon group having 1 to 26 carbon atoms, R³ represents a hydrocarbon group having 7 to 18 carbon atoms, and n is 1 or 2.

The hydrocarbon group represented by R¹ and R² may be any of linear, branched, cyclic, or a combination thereof. As the hydrocarbon group represented by R¹ and R², a hydrocarbon group containing an unsaturated bond may be used, however, it is preferable to use an alkyl group. As the hydrocarbon group represented by R¹ and R², hydrocarbon groups having 1 to 10 carbon atoms are preferable, linear or branched alkyl groups having 2 to 6 carbon atoms are more preferable, linear or branched alkyl groups having 3 to 5 carbon atoms are further preferable, and a n-butyl group is most preferable.

As the hydrocarbon group represented by R³, a hydrocarbon group containing an unsaturated bond may be used, however, it is preferable to use a saturated hydrocarbon group. From the standpoint of easiness of production, the hydrocarbon group represented by R³ is preferably a hydrocarbon group having 7 to 15 carbon atoms, more preferably a linear or branched alkyl group having 7 to 12 carbon atoms.

n is preferably 2. Therefore, the component (A) is preferably an N-acylglutamic acid derivative.

As the component (A), specifically, N-octanoyl-L-glutamic acid dibutylamide, N-2-ethylhexanoyl-L-glutamic acid dibutylamide, N-decanoyl-L-glutamic acid dibutylamide, N-lauroyl-L-glutamic acid dibutylamide, N-myristoyl-L-glutamic acid dibutylamide, N-palmitoyl-L-glutamic acid dibutylamide, N-stearoyl-L-glutamic acid dibutylamide and the like, can be used, and of them, N-2-ethylhexanoyl-L-glutamic acid dibutylamide and N-lauroyl-L-glutamic acid dibutylamide are preferably used, and it is preferable particularly to use both the compounds simultaneously from the standpoint of getting sufficient transparency.

In the oily composition of the present invention, the content of the component (A) is usually 0.1 to 25 wt %, preferably 0.5 to 15 wt %, more preferably 1 to 10 wt %. When the content of the component (A) is less than 0.1 wt %, gel strength is insufficient in some cases, and when the content of the component (A) is over 25 wt %, sufficient transparency cannot be gotten in some cases.

[Regarding Component (B)]

The component (B) is a polyamide resin. More specifically, it is as described below.

[1] It is preferable to use an amide-terminal polyamide resin represented by the following formula (2).

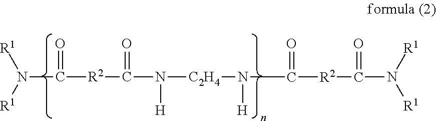

Specifically, “HAIMALATE PAM” (trade name; manufactured by Kokyu Alcohol Kogyo Co., Ltd.) can be used, and this is a (dimer dilinoleic acid bisdioctadecylamide/ethylenediamine) copolymer, wherein each R¹ is mutually the same or different and is linear or branched alkyl groups having 8 to 22 carbon atoms, each R² is dimer acid residues or dibasic acid residues, and n is 2 to 4 in the formula (2).

Further, “SYLVACLEAR A200V” (trade name; manufactured by Arizona Chemical Company) or “SYLVACLEAR A2614V” (trade name; manufactured by Arizona Chemical Company) can be used, in which each R¹ is alkylamine group residues having 14 to 18 carbon atoms, each R² is dimer dilinoleic acid residues, and n is 2 to 4 in the formula (2).

[2] It is preferable to use an ester-terminal polyamide resin represented by the following formula (3).

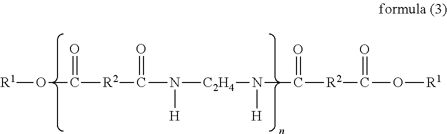

Specifically, “UNICLEAR 100VG” (trade name; manufactured by Arizona Chemical Company) can be used, in which each R¹ is stearyl alcohol residues, each R² is dimer dilinoleic acid residues, and n is 3 or 4 in the formula (3).

[3] As other polyamide resins, for example, “VERSAMID 930” (trade name; manufactured by Cognis) can be used.

Particularly, as the component (B), amide-terminal polyamide resins are preferable, and it is more preferable to use polyamide resins represented by the formula (2) (for example, “HAIMALATE PAM” (trade name; manufactured by Kokyu Alcohol Kogyo Co., Ltd.)) from the standpoint of getting sufficient transparency.

In the oily composition of the present invention, the content of the component (B) is usually 0.1 to 40 wt %, preferably 1 to 30 wt %, more preferably 2.5 to 25 wt %. When the content of the component (B) is less than 0.1 wt %, a sufficient pack feeling cannot be obtained in some cases, and when the content of the component (B) is over 40 wt %, elongation is poor in some cases.

Regarding the compounding ratio of the component (A) to the component (B) in the oily composition of the present invention, the ratio of the component (B) is usually 0.004 to 400 parts by weight, preferably 0.067 to parts by weight, more preferably 0.4 to 4 parts by weight with respect to 1 part by weight of the component (A). In the case of excess over the above-described range, it is difficult to get sufficient transparency in some cases, and a use feeling tends to deteriorate when processed into a preparation.

[Regarding Component (C)]

The component (C) is a liquid oil.

As the liquid oil, oils presenting liquid state at 25° C. are preferably used, and specifically, higher alcohols, amino acid derivatives, hydrocarbons, animal and plant-derived fats and oils, hardened oils, fatty acids, ester oils, lanolin.lanolin derivatives, lower alcohols, polyalcohols, fluorine oils, silicone oils, dimer acid esters, dimer diol esters, dimer diol ethers, fatty acid polyglyceryl and the like which are usually used in the art, can be used, and of them, higher alcohols, hydrocarbons, ester oils, lanolin.lanolin derivatives, silicone oils, animal and plant-derived fats and oils, dimer acid esters, dimer diol esters and dimer diol ethers can be preferably used.

More specifically, these are as described below.

[1] Higher Alcohol

Specifically, 2-octyldodecanol, 2-decyltetradecanol, isostearyl alcohol, 2-hexyldecanol, oleyl alcohol and the like can be used.

[2] Hydrocarbon

Specifically, light liquid isoparaffin, heavy liquid isoparaffin, liquid paraffin, polybutene, squalane, α-olefin oligomer, isohexadecane, isododecane, polyisobutene, liquid isoparaffin and the like can be used.

[3] Ester Oil

Specifically, diisostearyl malate, pentaerythrityl tetra(2-ethylhexanoate), neopentyl glycol dicaprate, pentaerythrityl tetraisostearate, glyceryl tri(2-ethylhexanoate), trimethylolpropane tri(2-ethylhexanoate), glyceryl triisostearate, trimethylolpropane triisostearate, pentaerythrityl hydrogenated rosinate, isotridecyl isononanoate, isononyl isononanoate, cetyl 2-ethylhexanoate, polyglyceryl isostearate and the like can be used.

[4] Lanolin.Lanolin Derivative

Specifically, liquid lanolin and the like can be used.

[5] Silicone Oil

Specifically, methylphenylpolysiloxane and the like can be used.

[6] Animal and plant-derived fats and oils

Specifically, macadamia nut oil, avocado oil, rosehip oil, olive oil, castor oil, sunflower oil, jojoba oil, meadowfoam oil and the like can be used.

[7] Dimer Acid Ester

Specifically, phytosteryl/isostearyl/cetyl/stearyl/behenyl dimer dilinoleate, diisopropyl dimerate, dimer dilinoleyl dimer dilinoleate, dimer dilinoleic acid hydrogenated castor oil, bis-phytosteryl/behenyl/isostearyl dimer dilinoleyl dimer dilinoleate, di-isostearyl/phytosteryl dimer dilinoleate and the like can be used.

[8] Dimer Diol Ester

Specifically, dimer dilinoleyl hydrogenated rosin condensate, dimer dilinoleyl diisostearate and the like can be used.

[9] Dimer Diol Ether

Specifically, hydroxyalkyl(C12-14) hydroxy dimer dilinoleyl ether and the like can be used.

In the oily composition of the present invention, the content of the component (C) is usually 40 to 95 wt o, preferably 50 to 90 wt %, more preferably 65 to 90 wt % from the standpoint of transparency and use feeling.

The oily composition of the present invention may optionally contain solid or semi-solid oils, active ingredients according to applications, and additional components, in addition to the above-described components (A), (B) and (C).

[Regarding Solid or Semi-Solid Oil]

As the solid or semi-solid oil, oils presenting solid or semi-solid state (not liquid) at 25° C. are preferably used, and specifically, higher alcohols, amino acid derivatives, hydrocarbons, animal and plant-derived fats and oils, hardened oils, fatty acids, ester oils, lanolin.lanolin derivatives, fluorine oils, silicone oils, waxes, hardened oils, fatty acids, dimer acid esters and the like which are usually used in the art, can be used, and of them, amino acid derivatives, hydrocarbons, lanolin.lanolin derivatives, waxes and dimer acid esters can be preferably used.

More specifically, these are as described below.

[1] Amino Acid Derivatives

Specifically, di-phytosteryl.behenyl.2-octyldodecyl N-lauroyl-L-glutamate and the like can be used.

[2] Hydrocarbon

Specifically, vaseline and the like can be used.

[3] Lanolin.lanolin Derivative

Specifically, lanolin and the like can be used.

[4] Waxes

Specifically, candelilla wax resin and the like can be used.

[5] Dimer Acid Ester

Specifically, phytosteryl/isostearyl/cetyl/stearyl/behenyl dimer dilinoleate, bis-phytosteryl/behenyl/isostearyl dimer dilinoleyl dimer dilinoleate, dimer dilinoleic acid hydrogenated castor oil and the like can be used.

It is preferable that the oily composition of the present invention contains substantially no solid oil from the standpoint of getting sufficient transparency.

[Regarding Active Ingredient]

Specifically, these are as described below.
[1] Anti-inflammatory components: stearyl glycyrrhetinate, salicylic acid, allantoin and the like;
[2] Vitamin components: tocopherol acetate, vitamin A oil and the like;
[3] Moisturizing ingredient: sodium hyaluronate, honey, royal jelly extract, whey, water-soluble collagen, ceramide and the like;
[4] Ultraviolet absorbing components: 2-ethylhexyl p-methoxycinnamate and the like;
[5] Analgesic components: menthol, camphor, salicylic acid, eucalyptus oil, methyl salicylate and the like;
[6] Antibacterial components: 1,2-pentanediol, 1,2-hexanediol, 1,2-octanediol and the like;
[7] Antiviral components.

[Regarding Additional Components]

Specifically, coloring agents, antiseptic agents, antifungal agents, antioxidants, sequestering agents, gloss agents, pH regulators, flavoring agents (including sweetening agent), correctives (including perfume), glossy powders and the like can be used.

[Regarding Solidification Point of Oily Composition]

The solidification point of the oily composition of the present invention is usually 70° C. or lower, preferably 68° C. or lower, particularly preferably 65° C. or lower from the standpoint of resolution of troubles in a preparation step and resolution of limitation of the material of a container due to the heat resistance of the container. The lower limit of the solidification point is usually 40° C. or higher, preferably 45° C. or higher, particularly preferably 50° C. or higher from the standpoint of use as a rod-shaped article.

[Regarding Hardness of Oily Composition]

The hardness of the oily composition of the present invention after solidification is usually 10 to 100 (g/0.5×0.5×3.14 mm²), preferably 15 to 90 (g/0.5×0.5×3.14 mm²), more preferably 25 to 85 (g/0.5×0.5×3.14 mm²).

A rheometer (trade name “NRM-2002)”, manufactured by Fudou Kougyou Inc.) is used, a cylindrical adapter having a diameter of 1 mm (adapter No. 6) is allowed to go into through a halfway position between the axis center and the inner periphery of a tubular container at a rate of 20 mm/min, and the maximum hardness value during 30 seconds from the ingress means the hardness in the present invention (unit: g/0.5×0.5×3.14 mm²).

(Rod-Shaped Preparation)

The rod-shaped preparation of the present invention is obtained by solidification of the above-described oily composition of the present invention into a rod-shape.

For fabrication of the rod-shaped preparation of the present invention, the oily composition is melted by heating, then, filled in a tubular container and cooled.

As the tubular container to be used for the rod-shaped preparation of the present invention, known tubular containers can be used without particular restriction, and feeding containers widely used for lip rouges, lip creams and the like are preferably used from the standpoint of convenience of users.

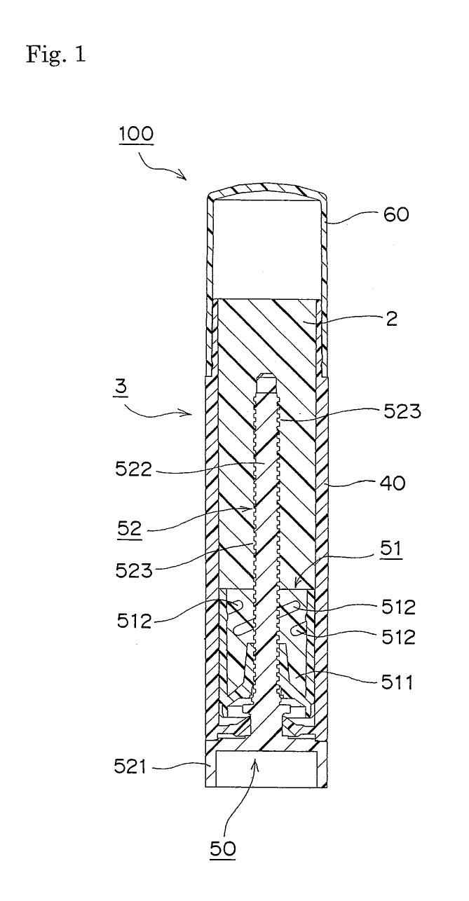

FIG. 1 is a sectional view showing a rod-shaped article containing the rod-shaped preparation of the present invention. This rod-shaped article 100 is equipped with a rod-shaped preparation 2 and a feeding container 3. The feeding container 3 is equipped with a tubular container 40 for storing the rod-shaped preparation 2, a feeding mechanism 50 for feeding the rod-shaped preparation 2 stored in the container 40 out of the container 40, and a cap 60 for covering the container 40. The cap 60 is removable from the container 40.

The container 40 has a circular cross-section. The rod-shaped preparation 2 is slidably engaged in the container 40.

The feeding mechanism 50 is equipped with a bucket 51 and a rotation axis 52. The bucket 51 has a concave portion 511 in which the rod-shaped preparation 2 is filled. The rotation axis 52 has a dial 521 exposed to the bottom and a center axis 522 coming up vertically into the container 40 from the dial 521. The center axis 522 has a lot of grooves 523 on its surface. The bucket 51 is threadably mounted on the grooves 523 of the center axis 522, and when the center axis 522 rotates, the bucket 51 moves up and down along the center axis 522. According to the feeding mechanism 50, the center axis 522 rotates owing to rotation of the dial 521, followed by movement of the bucket 51 with the rod-shaped preparation 2 up and down in the container 40, thereby feeding the rod-shaped preparation 2.

The rod-shaped article 100 is used while feeding the rod-shaped preparation 2 in the container 40 out of the container 40 by the feeding mechanism 50. Here, “feeding” means “drawing out and in” of the rod-shaped preparation 2 from and into the container 40.

The above-described rod-shaped article 100 is produced via the following steps (1) and (2) in this order:

(1) a step of preparing the oily composition of the present invention, and

(2) a molding step of melting the above-described oily composition by heating, filling the melted material into the container 40, and solidifying this into a rod-shape by cooling.

The bucket 51 has ribs 512 spirally extending on the inner periphery. In the drawing, the ribs 512 are formed in four rows. The molded rod-shaped preparation 2 does not come away easily from the bucket 51 owing to the ribs 512. Thus, the rod-shaped preparation 2 is fed from the container 40 stably. The ribs 512 are formed not on the whole inner periphery of the bucket 51 but formed only on the upper inner periphery. If the ribs 512 are formed on the whole inner periphery of the bucket 51, the rod-shaped preparation 2 in the bucket 51 becomes fragile. In the rod-shaped article 100, however, the ribs 512 are formed only on the upper inner periphery of the bucket 51, thus, it is possible to suppress the rod-shaped preparation 2 in the bucket 51 from becoming fragile. The number of the rows of the ribs to be formed is not particularly restricted.

According to the above-described method of manufacturing the rod-shaped article 100, the productivity of the rod-shaped article 100 can be improved since the rod-shaped preparation 2 is formed directly in the container 40.

The tubular container, the feeding mechanism (bucket and rotation axis) and the cap used in the rod-shaped article of the present invention are generally constituted of hard synthetic resins such as ABS, AS, polypropylene, polyethylene, polyethylene terephthalate, polyethylene naphthalate, polyester, polyvinyl chloride, polycarbonate and the like, and it is preferable to use hard synthetic resins having a heat resistance of 75° C. or higher. As such hard synthetic resins having a heat resistance of 75° C. or higher, specifically, ABS, AS, polypropylene and the like can be used.

Particularly, when the oily composition of the present invention has excellent transparency, it is preferable to use a tubular container made of a resin having high transparency. Regarding the transparency of the tubular container, the total light transmittance (JIS K 7361-1/2 mmt) is 70% or more, preferably 80% or more, more preferably 85% or more, particularly preferably 90% or more. Specifically, those made of an ABS resin having high transparency (for example, the total light transmittance (JIS K 7361/2 mmt) is 90% or more) are preferable as the tubular container. For the feeding mechanism, the bucket is preferably made of polypropylene, and the rotation axis is preferably made of polypropylene or an ABS resin having high transparency. The cap is preferably made of polypropylene.

### EXAMPLES

Oily compositions of Examples 1 to 4 of the present invention and oily compositions of Comparative Examples 1 to 4 were prepared. Components contained in these oily compositions are as described in Table 1. That is, the oily compositions of Examples 1 to 4 contain all of the above-described components (A), (B) and (C), while the oily compositions of Comparative Examples 1 to 4 do not contain the component (B).

TABLE 1

| | | examples | | | | comparative examples | | | |
|---|---|---|---|---|---|---|---|---|---|
| component | specific examples | 1 | 2 | 3 | 4 | 1 | 2 | 3 | 4 |
| A | N-2-ethylhexanoyl-L-glutamic acid | 2 | 3 | 3 | 3 | 2 | 6 | 2 | 2 |
| | dibutylamide | | | | | | | | |
| | N-lauroyl-L-glutamic acid | 3 | 5 | 5 | 5 | 3 | 9 | 3 | 3 |
| | dibutylamide | | | | | | | | |
| B | (dimer dilinoleic acid | 5 | 10 | 10 | 10 | — | — | — | — |
| | bisdioctadecylamide/ethylenediamine) | | | | | | | | |
| | copolymer | | | | | | | | |
| | 12-hydroxystearic acid | — | — | — | — | 5 | — | — | — |
| | dextrin palmitate | — | — | — | — | — | 3 | — | — |
| | polyglyceryl deca(behenate/caprate) | — | — | — | — | — | — | 5 | — |
| C | 2-octyldodecanol | 20 | 30 | 30 | 30 | 20 | 50 | 20 | 20 |
| | liquid paraffin | 25 | 22 | 22 | 22 | 25 | — | 25 | 30 |
| | diisostearyl malate | 15 | 10 | 10 | 10 | 15 | — | 15 | 45 |
| | dimer dilinoleyl dimer dilinoleate | 30 | 20 | — | — | — | 10 | — | — |
| | hydroxyalkyl(C12-14) hydroxy dimer | — | — | — | — | 30 | — | — | — |
| | dilinoleyl ether | | | | | | | | |
| | hydrogenated polyisobutene | — | — | 20 | — | — | — | 30 | — |
| | polybutene | — | — | — | 20 | — | — | — | — |

| | | | | | | | | |
|---|---|---|---|---|---|---|---|---|
| solidification point (° C.) | 61 | 66 | 78 | 76 | | — | — | — |
| melting point (° C.) | 66 | 81 | 86 | 86 | | — | — | — |
| hardness | 30.8 | 52 | 48 | 48 | | — | — | — |
| transparency/transmittance (600 nm) | 94.4 | 89.6 | 92.7 | 92.7 | 74.6 | 21.0 | 0.5 | 80.8 |
| evaluation from transparency/transmittance | ⊚ | ⊚ | ⊚ | ⊚ | Δ | X | X | ◯ |
| use feeling (sliding property)/friction | 0.89 | 0.86 | 1.05 | 0.90 | 1.03 | 1.25 | 1.15 | 1.93 |
| sensitivity tester | | | | | | | | |
| evaluation from use feeling (sliding | ⊚ | ⊚ | ◯ | ⊚ | ◯ | Δ | Δ | X |
| property)/friction sensitivity tester | | | | | | | | |

In Table 1, units of numerical values showing compounding ratios of components are parts by weight.

The oily compositions of Examples 1 to 4 and Comparative Examples 1 to 4 were melted by heating, then, filled in a tubular container and cooled, thereby obtaining rod-shaped preparations. Various physical properties, transparency and use feeling were measured by using these rod-shaped preparations.

[Regarding Physical Properties]

(1) Solidification Point

Five grams (5 g) of a rod-shaped preparation was crushed finely and charged in a 20 ml screw cap bottle. Then, a suitable amount of silicone oil was charged in a stainless bat, the bat was placed on a hot plate (trade name “ECHOTPLATE EC-1200N”, manufactured by ASONE Corporation) and heated, the screw cap bottle containing the above-described preparation was placed in the bat, and the above-described preparation was melted completely, then, allowed to stand at room temperature while keeping the condition unchanged. The temperature when solidification of the preparation was confirmed by a micro-spatula was adopted as the solidification point. As the thermometer, “CUSTOM CT-450WR” (trade name: manufactured by CUSTOM) was used.

(2) Melting Point

Five grams (5 g) of a rod-shaped preparation was crushed finely and charged in a 20 ml screw cap bottle. Then, a suitable amount of silicone oil was charged in a stainless bat, the bat was placed on a hot plate (trade name “ECHOTPLATE EC-1200N”, manufactured by ASONE Corporation) and heated, the screw cap bottle containing the above-described preparation was placed in the bat, and the appearance of the above-described preparation was observed visually and the temperature when the above-described preparation started to melt was adopted as the melting point. As the thermometer, “CUSTOM CT-450WR” (trade name: manufactured by CUSTOM) was used.

(3) Hardness

As the above-described tubular container to be filled with an oily composition, a cylindrical container having a diameter of about 13 mm and a height of about 60 mm was used. For cooling after filling, a constant temperature of 25° C. was kept for 12 hours or more. Ten millimeters (10 mm) of an upper part of the resultant rod-shaped preparation was cut, and the residual rod-shaped preparation was allowed to descend into the above-described tubular container. This was used as a sample for measurement. Its hardness was measured by using a rheometer (trade name “NRM-2002)”, manufactured by Fudou Kougyou Inc.). That is, a cylindrical adapter having a diameter of 1 mm (adapter No. 6) was allowed to go into through a halfway position between the axis center and the inner periphery of the above-described tubular container at a rate of 20 mm/min, and the maximum hardness value during 30 seconds from the ingress was adopted as the hardness (unit: g/0.5×0.5×3.14 mm²).

[Regarding Transparency]

As the above-described tubular container to be filled with an oily composition, a cell having an optical path length of 10 mm (cell size: 10×10×45 mm, bilateral transmission type, made of polystyrene) was used. The cooling-solidified rod-shaped preparation was allowed to stand at 25° C. for 1 hour, and this was used as a sample for measurement. The transmittance of 600 nm through the above-described preparation in the above-described cell was measured by using a spectrophotometer (trade name “UV-2450”, manufactured by Shimadzu Corp.). In Table 1, 85 or more is represented by “⊚”, 75 or more and less than 85 is represented by “◯”, 50 or more and less than 75 is represented by “Δ”, and less than 50 is represented by “×”.

[Regarding Use Feeling]

(1) “Elongation”

On an area of 10 cm² (5 cm×2 cm) on the surface of an acrylic plate, 20 mg of the above-described preparation was applied. The dynamic friction coefficient with respect to the above-described preparation was measured by using a static and dynamic friction measuring machine Tribo-master (trade name “TL201S”, manufactured by Trinity Laboratory Co., Ltd.). That is, a friction sensitivity tester was moved at a rate of 2.0 mm/sec under a load of 100 g, and by this, the dynamic friction coefficient was measured. The measured data was transferred to a computer by using Condition catcher Model (trade name “MPL-10A-128”, manufactured by Kyushukyohan Co., Ltd.), and the average value of the dynamic friction coefficient was calculated in the computer.

The average value of the dynamic friction coefficient shows “sliding property” of the above-described preparation, and this “sliding property” and “elongation” have a correlation, thus, “elongation” can be evaluated based on the average value of the dynamic friction coefficient. Smaller the average value of the dynamic friction coefficient, better the “sliding property”. In Table 1, less than 1.00 is represented by “⊚”, 1.00 or more and less than 1.10 is represented by “◯”, 1.10 or more and less than 1.20 is represented by “Δ”, and 1.20 or more is represented by “×”.

(2) “Pack Feeling”

An evaluation was performed for Examples 1 to 4 and Comparative Example 1. The evaluation was performed by 10 monitors based on 5 step points. “Significant positive” is evaluated as 5 points, “positive” is evaluated as 4 points, “usual” is evaluated as 3 points, “negative” is evaluated as 2 points and “complete negative” is evaluated as 1 point, thus calculating the total point. The results are shown in Table 2.

TABLE 2

| | significant | | | | complete | |
|---|---|---|---|---|---|---|
| | positive | positive | usual | negative | negative | total |
| Example 1 | 5 | 1 | 3 | 1 | 0 | 40 |
| Example 2 | 2 | 3 | 1 | 4 | 0 | 33 |
| Example 3 | 0 | 2 | 2 | 6 | 0 | 26 |
| Example 4 | 1 | 3 | 3 | 1 | 1 | 29 |
| Comparative | 0 | 1 | 2 | 6 | 1 | 23 |
| Example 1 | | | | | | |

[Consideration]

(1) The preparations obtained by solidification of the oily compositions of Examples 1 to 4 have degrees of transparency of 85 or more and are excellent in transparency as compared with Comparative Examples 1 to 3 using oily gelling agents widely used in the art (12-hydroxystearic acid, dextrin palmitate, polyglycerin fatty acid ester) and Comparative Example 4 using only the component (A). The reason for this is believed as inclusion of a polyamide resin as the component (B). Therefore, the oily composition of the present invention is, when solidified and used as a preparation, capable of sufficiently exerting an advantage of transparency.

(2) The preparations obtained by solidification of the oily compositions of Examples 1 to 4 are excellent also in use feelings such as “elongation” and “pack feeling”. The reason for this is believed as inclusion of a polyamide resin as the component (B). Therefore, the oily composition of the present invention is, when solidified and used as a preparation, capable of providing a satisfactory use feeling.

(3) The preparations obtained by solidification of the oily compositions of Examples 1 and 2 are excellent particularly in “pack feeling”. The reason for this is believed as inclusion of a dimer acid ester as the component (C).

(4) Since the oily compositions of Examples 1 to 4 have low solidification points, solidification during a preparation step can be prevented, thus, handling thereof is easy.

Specific examples of the rod-shaped preparation of the present invention are shown in Table 3. As specific examples, two “lip rouges”, two “stick-form lip glosses” and three “transparent lip creams” are shown.

TABLE 3

| | lip | lip | stick-form | stick-form | transparent | transparent | transparent |
|---|---|---|---|---|---|---|---|
| component | rouge 1 | rouge 2 | lip gloss 1 | lip gloss 2 | lip cream 1 | lip cream 2 | lip cream 3 |
| N-2-ethylhexanoyl-L-glutamic acid | 5 | 3 | 3 | 3 | 2 | 2 | — |
| dibutylamide | | | | | | | |
| N-lauroyl-L-glutamic acid | 5 | 5 | 5 | 5 | 3 | 3 | 10 |
| dibutylamide | | | | | | | |
| (dimer dilinoleic acid | 5 | 8 | 5 | 5 | 10 | 15 | 10 |
| bisdioctadecylamide/ethylenediamine) | | | | | | | |
| copolymer | | | | | | | |
| 2-octyldodecanol | 30 | 20 | 30 | 30 | 20 | 20 | 30 |
| diisostearyl malate | residue | 25 | 15 | 15 | 10 | residue | 10 |
| dimer dilinoleyl dimer dilinoleate | 15 | 15 | — | — | 10 | — | 20 |
| (trade name “LUSPLAN DD-DA5”, | | | | | | | |
| manufactured by Nippon Fine Chemical | | | | | | | |
| Co., Ltd.) | | | | | | | |
| pentaerythrityl | — | residue | — | — | — | — | — |
| tetra(2-ethylhexanolate) | | | | | | | |
| methylphenylpolysiloxane | 5 | — | 12 | 12 | — | — | — |
| hydroxyalkyl(C12-14) hydroxy dimer | — | — | — | 1 | — | — | — |
| dilinoleyl ether | | | | | | | |
| polyglyceryl isostearate | — | — | — | — | — | 3 | — |
| heavy liquid isoparaffin | — | — | residue | — | — | — | — |
| (trade name “PARMLEAM 24”, | | | | | | | |
| manufactured by NOF Corporation) | | | | | | | |
| heavy liquid isoparaffin | — | — | — | residue | — | — | — |
| (trade name “PARMLEAM 18”, | | | | | | | |
| manufactured by NOF Corporation) | | | | | | | |
| liquid paraffin | — | — | — | — | residue | — | 20 |
| liquid lanolin | — | — | — | — | 20 | 20 | — |
| macadamia nut oil | — | — | — | — | 3 | — | — |
| meadowfoam oil | — | — | — | — | — | 3 | — |
| squalane | — | — | — | — | 3 | 3 | — |
| red No. 223 | 0.2 | 0.1 | — | — | — | — | — |
| red No. 218 | — | 0.1 | — | — | — | — | — |
| red No. 202 | 0.8 | — | — | — | — | — | — |
| mica titanium | — | — | 1 | 1 | — | — | — |
| perfume | s.a.* | s.a.* | s.a.* | s.a.* | s.a.* | s.a.* | s.a.* |
| antioxidant | s.a.* | s.a.* | s.a.* | s.a.* | s.a.* | s.a.* | s.a.* |
| tocopherol acetate | — | — | — | — | s.a.* | s.a.* | s.a.* |
| vitamin A oil | — | — | — | — | s.a.* | s.a.* | s.a.* |

s.a.* = suitable amount

### INDUSTRIAL APPLICABILITY

The oily composition of the present invention is, upon solidification to give a preparation for lip, capable of exhibiting sufficient transparency and further capable of providing a satisfactory use feeling, thus, its industrial use value is significant.

1. An oily composition which gives a preparation for lip through solidification, characterized by comprising:
(A) an N-acylamino acid derivative represented by the following formula (1),
(B) a polyamide resin, and
(C) a liquid oil: 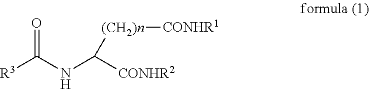
(in the formula (1), R¹ and R² represent each independently a hydrocarbon group having 1 to 26 carbon atoms, R³ represents hydrocarbon group having 7 to 18 carbon atoms, and n is 1 or 2.).

2. The oily composition according to claim 1, comprising a (dimer dilinoleic acid bisdioctadecylamide/ethylenediamine) copolymer as the component (B).

3. The oily composition according to claim 1 or 2, comprising two N-acylglutamic acid dialkylamides as the component (A).

4. The oily composition according to claim 1, comprising a dimer acid ester as the component (C).

5. A rod-shaped preparation, characterized in that it is obtained by solidification of the oily composition according to claim 1 into a rod-shape.
